(19) United States
(12) Patent Application Publication
(10) Pub. No.: US 2025/0273923 A1
(43) Pub. Date: Aug. 28, 2025

(54) LASER CAVITY SYSTEMS FOR VARIOUS SENSING CONFIGURATIONS

(71) Applicant: DeUVe Photonics, Inc., Tucson, AZ (US)

(72) Inventors: Chris Hessenius, Tucson, AZ (US); Mahmoud Fallahi, Tucson, AZ (US)

(21) Appl. No.: 19/059,729

(22) Filed: Feb. 21, 2025

Related U.S. Application Data
(60) Provisional application No. 63/557,803, filed on Feb. 26, 2024.
(60) Provisional application No. 63/572,594, filed on Apr. 1, 2024.

(51) Int. Cl. H01S 5/00; H01S 5/183

(52) U.S. Cl. CPC H01S 5/0014; H01S 5/183

(57) ABSTRACT

A laser device for measuring one or more properties of an object adjacent to the device. The device may comprise an open laser cavity comprising one or more gain chips disposed at the first end of the open laser cavity, configured to generate one or more laser beams. The open laser cavity may further comprise one or more sensing media disposed at the second end of the open laser cavity and optically in line with the one or more gain chips. The one or more sensing media may comprise a proximal end having a transmissive coating and a distal end having a reflective coating, configured to accept the one or more laser beams, measure the one or more properties of the object based on illumination by the one or more laser beams, and reflect the one or more laser beams back to the one or more gain chips.

### CROSS-REFERENCE TO RELATED APPLICATIONS

This application is a non-provisional and claims benefits of U.S. Provisional Application No. 63/557,803 filed Feb. 26, 2024 and U.S. Provisional Application No. 63/572,594 filed Apr. 1, 2024, the specifications of which are incorporated herein in their entirety by reference.

### FIELD OF THE INVENTION

The present invention is directed to a laser cavity system for up-close imaging with multi-target and increased resolution capabilities.

### BACKGROUND OF THE INVENTION

For certain laser imaging applications, such as in vivo organ imaging during surgery, up-close measurement would be a useful configuration. However, present laser imaging applications are currently limited in measuring range due to the need for an end mirror within the system, preventing the sensing material from being placed close enough to the end of the cavity. Thus, there exists a present need for a laser imaging system capable of executing up-close imaging. Furthermore, there also exists a present need for a laser cavity system capable of implementing multiple lasers for imaging multiple objects or increasing spatial resolution.

### BRIEF SUMMARY OF THE INVENTION

It is an objective of the present invention to provide devices that allow for a laser cavity system for up-close imaging with multiple-target and increased-resolution capabilities, as specified in the independent claims. Embodiments of the invention are given in the dependent claims. Embodiments of the present invention can be freely combined with each other if they are not mutually exclusive.

The present invention features a laser device for measuring one or more properties of an object adjacent to the device. The device may comprise a laser cavity having a first end and a second end. The laser cavity may comprise one or more gain chips disposed at the first end of the laser cavity, configured to generate one or more laser beams. The laser cavity may further comprise one or more sensing media disposed at the second end of the laser cavity and optically in line with the one or more gain chips. The one or more sensing media may comprise a proximal end having a transmissive coating and a distal end having a reflective coating. The one or more sensing media may be configured to accept the one or more laser beams, measure the one or more properties of the object based on illumination by the one or more laser beams, and reflect the one or more laser beams back to the one or more gain chips.

One of the unique and inventive technical features of the present invention is the implementation of a sensing medium having a transmissive coating on one end and a reflective coating on the other. Without wishing to limit the invention to any theory or mechanism, it is believed that the technical feature of the present invention advantageously provides for up-close laser imaging. None of the presently known prior references or work has the unique inventive technical feature of the present invention.

Another one of the unique and inventive technical features of the present invention is the implementation of multiple laser portions directed to multiple laser sending locations. Without wishing to limit the invention to any theory or mechanism, it is believed that the technical feature of the present invention advantageously provides for imaging of multiple objects simultaneously and increased spatial resolution. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows an embodiment of the up-close imaging system of the present invention showing a single gain chip producing a single laser portion directed towards a single sensing medium to analyze a single object.

FIG. 1B shows an embodiment of the up-close imaging system of the present invention showing a single gain chip producing multiple laser portions directed towards a single sensing medium to analyze a single object.

FIG. 1C shows an embodiment of the up-close imaging system of the present invention showing a single gain chip producing multiple laser portions directed towards multiple sensing media to analyze a single object.

FIG. 1D shows an embodiment of the up-close imaging system of the present invention showing multiple gain chips producing multiple laser portions directed towards multiple sensing media to analyze a single object.

FIG. 1E shows an embodiment of the up-close imaging system of the present invention showing a laser cavity comprising optical components such that the gain chip and the sensing medium are on the same side.

FIG. 1F shows an embodiment of the up-close imaging system of the present invention showing a single gain chip producing multiple laser portions directed towards multiple sensing media to analyze multiple objects.

FIG. 2A shows an embodiment of the up-close imaging system of the present invention comprising a gain medium with a V-fold.

FIG. 2B shows an embodiment of the up-close imaging system of the present invention comprising a gain medium with a V-fold and multiple sensing media.

FIG. 2C shows an embodiment of the up-close imaging system of the present invention comprising a gain medium with a V-fold and multiple sensing media for analyzing multiple objects.

FIG. 2D shows another embodiment of the up-close imaging system of the present invention comprising multiple gain chips each generating a single laser portion, each laser portion directed towards a single sensing medium, the sensing media analyzing a single object.

FIG. 2E shows another embodiment of the up-close imaging system of the present invention comprising multiple gain chips each generating a single laser portion, each laser portion directed towards a single sensing medium, each analyzing a single object.

FIG. 3A shows an embodiment of the up-close imaging system of the present invention comprising a gain medium with a V-fold and a sensing medium with another V-fold.

FIG. 3B shows an embodiment of the up-close imaging system of the present invention comprising a gain medium with a V-fold and multiple sensing media, one of which has another V-fold.

FIG. 3C shows an embodiment of the up-close imaging system of the present invention comprising a gain medium with a V-fold and multiple sensing media, one of which has another V-fold, each sensing medium analyzing an object.

FIG. 3D shows an embodiment of the up-close imaging system of the present invention comprising multiple gain media directing multiple laser portions towards multiple sensing media, one of which comprising a V-fold.

FIG. 4A shows an embodiment of the system of the present invention comprising a plurality of gain media and a plurality of sensing media.

FIG. 4B shows an embodiment of the system of the present invention comprising a plurality of gain media and a single sensing medium.

FIG. 4C shows an embodiment of the system of the present invention comprising a plurality of gain media and a plurality of sensing media, one of which comprising a V-fold.

FIG. 5A shows a first embodiment of a higher-order laser profile.

FIG. 5B shows a second embodiment of a higher-order laser profile.

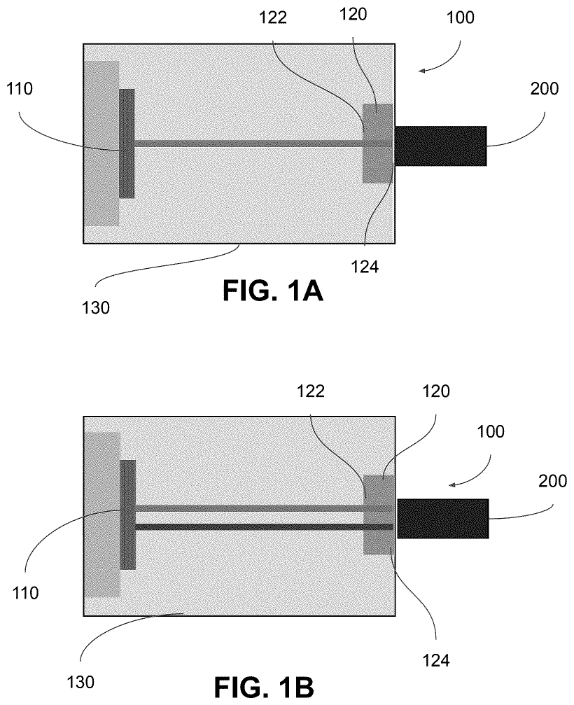

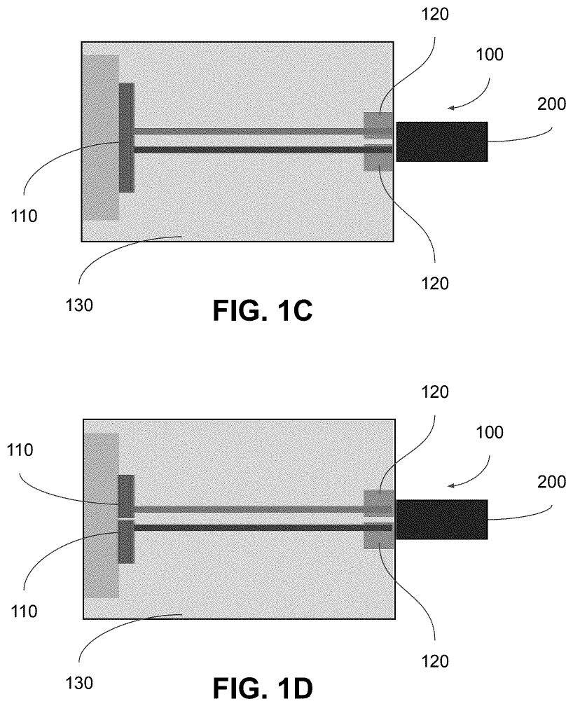

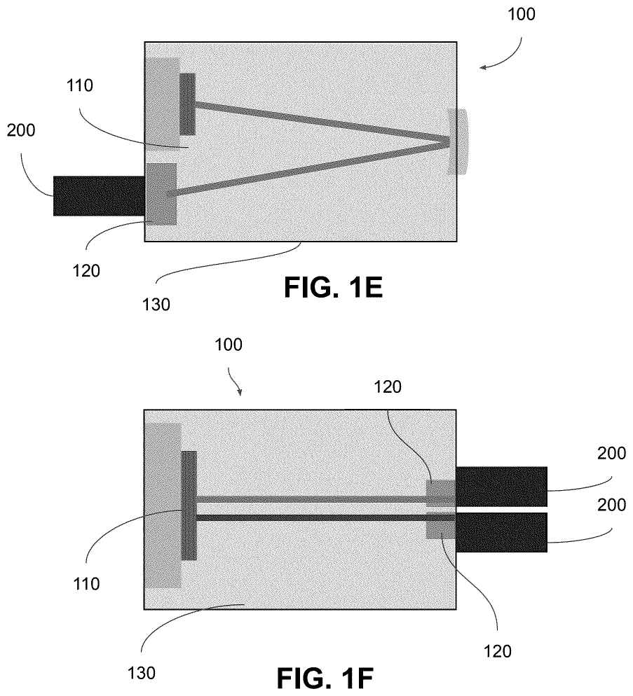

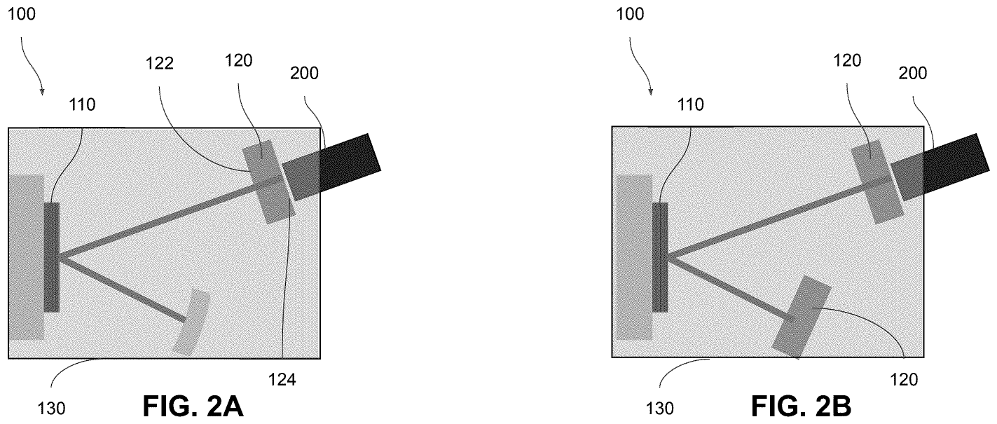

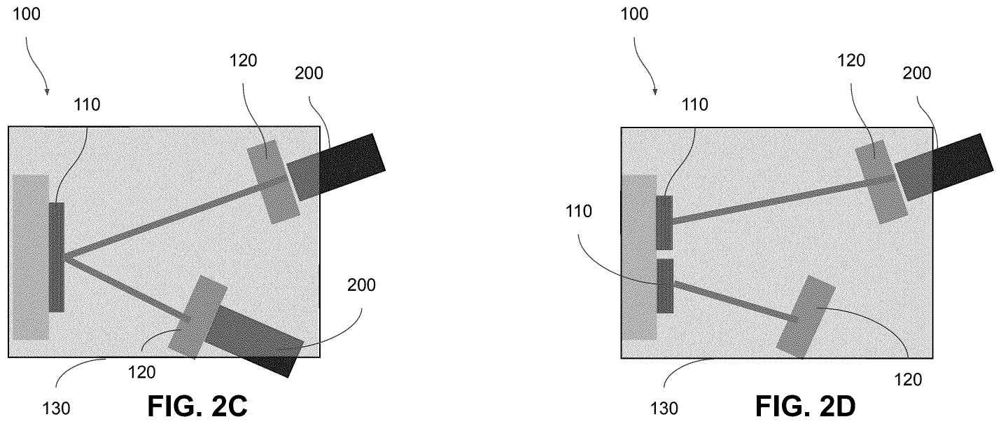

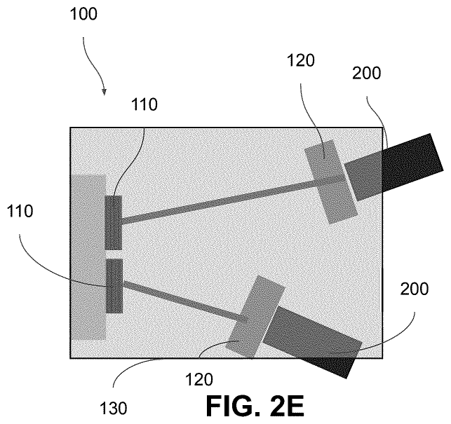

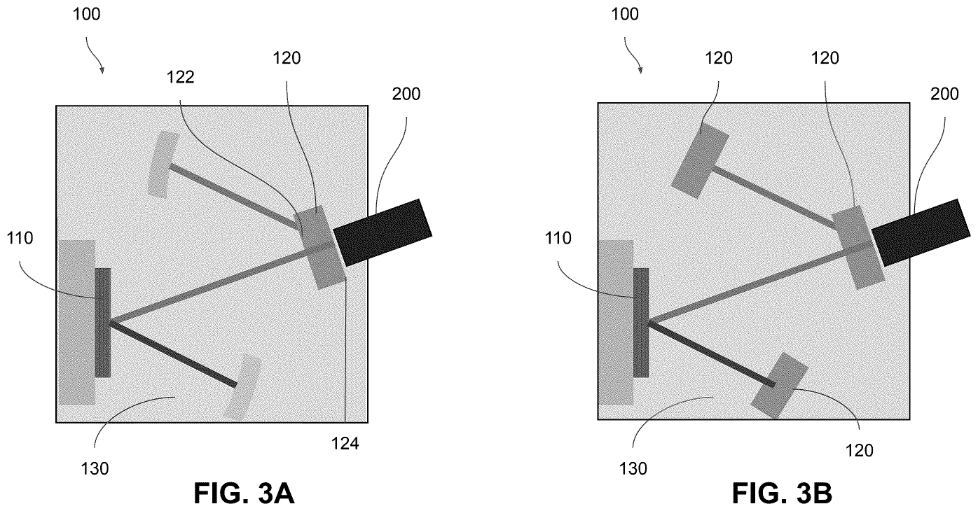

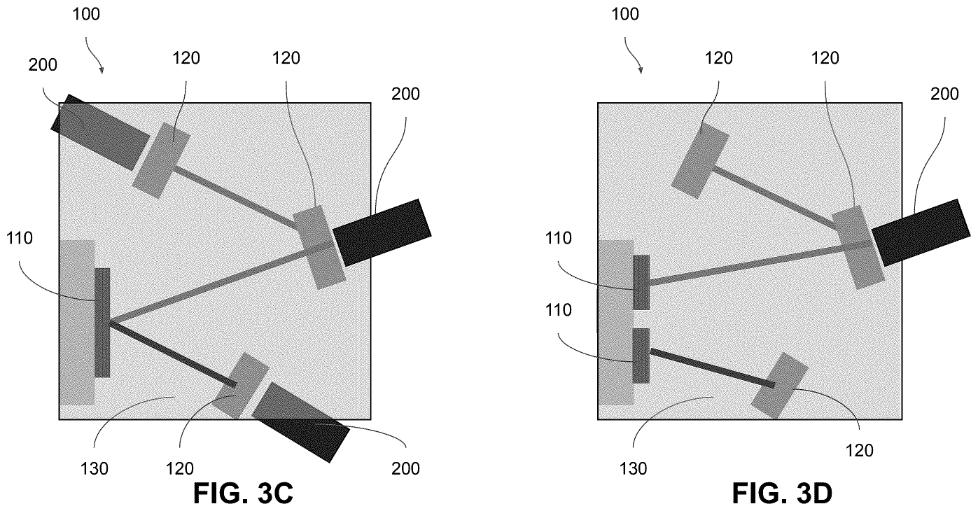

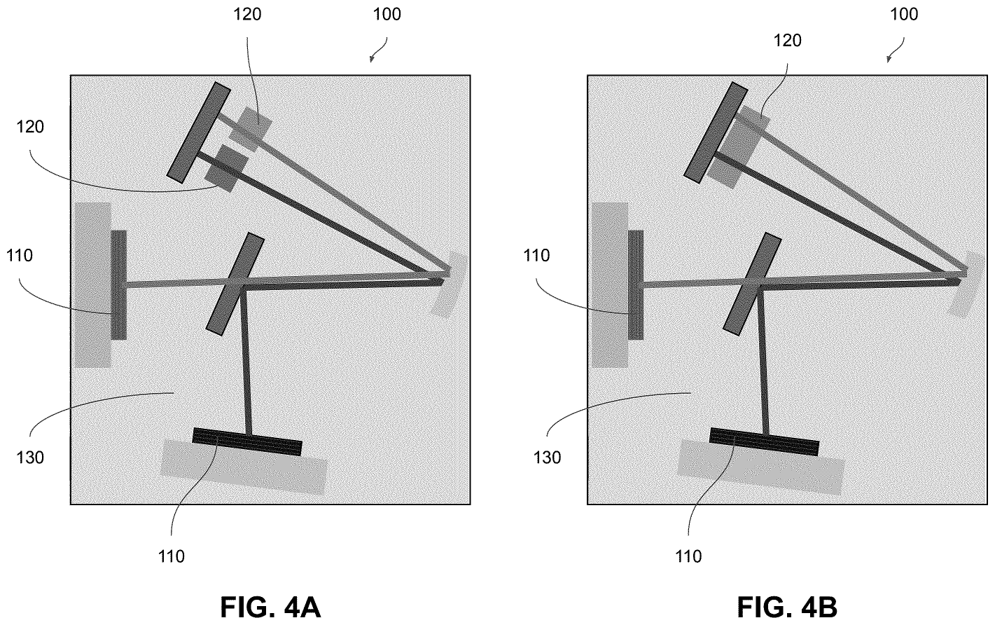

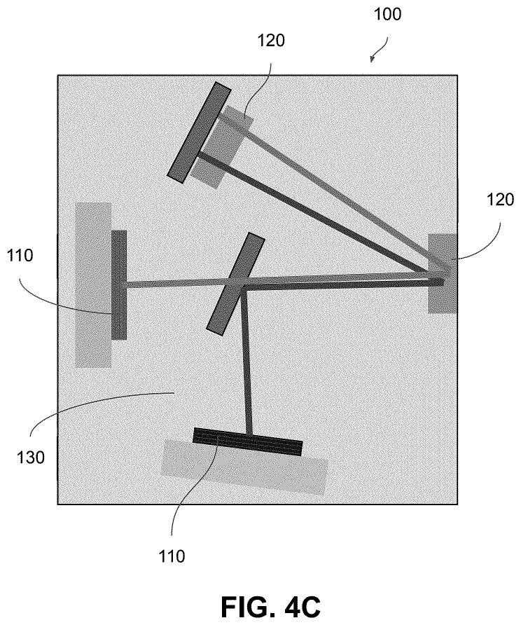

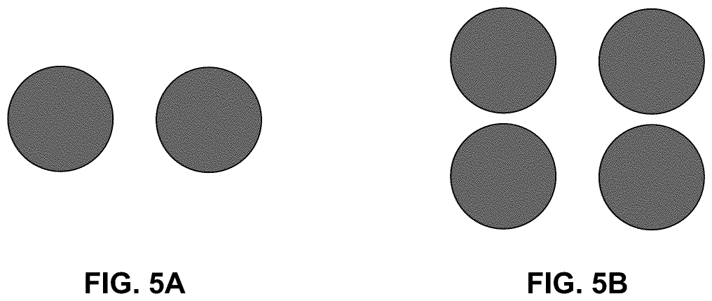

### DETAILED DESCRIPTION OF THE INVENTION

Following is a list of elements corresponding to a particular element referred to herein:
100 device
110 gain medium
120 sensing medium
122 proximal end
124 distal end
130 laser cavity
200 object

The term “sensing medium” is defined herein as a material configured to interact with one or more laser portions and interact with an external force such that a measurable change in the laser portion takes place.

The term “open laser cavity” is defined herein as a cavity configured to contain lasers that are not confined to their gain medium/media.

The present invention features a laser device (100) for measuring one or more properties of one or more objects (200). In some embodiments, the device (100) may comprise an open laser cavity (130) configured to contain one or more laser beams, each laser beam comprising one or more laser portions. The open laser cavity (130) may be configured to support multiple laser lines. The open laser cavity (130) may comprise one or more gain chips (110) disposed within the open laser cavity (130), configured to generate the one or more laser beams. The open laser cavity (130) may further comprise one or more sensing media (120) disposed within the open laser cavity (130) and optically in line with the one or more gain chips (110). Each sensing medium may comprise a proximal end (122) having a transmissive coating and a distal end (124) having a reflective coating. The one or more sensing media (120) may be configured to accept the one or more laser portions of the one or more laser beams, measure the one or more properties of the one or more objects (200) based on illumination by the one or more laser portions, and reflect the one or more laser portions of the one or more laser beams back to the one or more gain chips (110).

In some embodiments, at least one of the one or more gain chips (110) may comprise a vertical external-cavity surface-emitting laser. In some embodiments, the one or more sensing media (120) may be configured to interact with the one or more laser portions of the one or more laser beams and interact with an external force such that a measurable change in the one or more laser portions takes place. In some embodiments, the external force may comprise a magnetic field, temperature, humidity, vibration, altitude, or a combination thereof. In some embodiments, the measurable change may comprise laser power (e.g. the laser turning on/off), beam quality, line width, wavelength/color, phase output, or a combination thereof. Each sensing medium of the one or more sensing media (120) may be configured to support interactions with the laser as close to the one or more objects (200) as possible. In some embodiments, at least one of the one or more sensing media (120) may comprise a nitrogen-vacancy diamond, nitrogen-vacancy silicon carbide, or a combination thereof.

The up-close imaging system of the present invention may comprise any number of gain chips disposed at any point within the open laser cavity. The up-close imaging system of the present invention may comprise any number of sensing media disposed at any point within the open laser cavity optically in-line with the one or more gain chips. The up-close imaging system of the present invention may be configured to analyze any number of objects with any number of sensing media. The gain chip(s) of the up-close imaging system of the present invention may be configured to generate any number of laser beams, each laser beam comprising any number of laser portions. The open laser cavity may comprise any shape, any number of ends, and any number of bends. The one or more laser beams may comprise any beam shape and/or profile.

In some embodiments, at least one of the one or more gain chips (110) may comprise a V-fold configured to extend a path length of the one or more laser portions. In some embodiments, at least one of the one or more sensing media (120) may comprise a V-fold configured to extend a path length of the one or more laser portions. In some embodiments, the open laser cavity (130) may further comprise one or more optical elements. In some embodiments, the one or more laser beams may comprise a plurality of laser beams. The one or more gain chips (110) may comprise a plurality of gain chips such that each gain chip is configured to generate a laser beam of the plurality of laser beams.

In some embodiments, the one or more laser beams may comprise a laser beam having a higher-order laser mode profile. In some embodiments, the one or more sensing media (120) may comprise a plurality of sensing media such that at least one of the one or more laser portions is configured to interact with each sensing medium of the plurality of sensing media. In some embodiments, at least one of the one or more sensing media (120) may comprise a sensing medium comprising a plurality of laser interaction locations such that at least one of the one or more laser portions are configured to interact with each laser interaction location of the plurality of laser interaction locations.

The present invention features a laser device (100) for measuring one or more properties of one or more objects (200) adjacent to the device (100). In some embodiments, the device (100) may comprise an open laser cavity (130) having a first end and a second end. In some embodiments, the open laser cavity (130) may comprise one or more gain chips (110) disposed at the first end of the open laser cavity (130), configured to generate one or more laser beams. The open laser cavity (130) may further comprise one or more sensing media (120) disposed at the second end of the open laser cavity (130) and optically in line with the one or more gain chips (110). The one or more sensing media (120) may comprise a proximal end (122) having a transmissive coating and a distal end (124) having a reflective coating. The one or more sensing media (120) may be configured to accept the one or more laser beams, measure the one or more properties of the one or more objects (200) based on illumination by the one or more laser beams, and reflect the one or more laser beams back to the one or more gain chips (110).

In some embodiments, at least one of the one or more gain chips (110) may comprise a vertical external-cavity surface-emitting laser. In some embodiments, at least one of the one or more sensing media (120) may comprise a nitrogen-vacancy diamond, nitrogen-vacancy silicon carbide, or a combination thereof. In some embodiments, at least one of the one or more gain chips (110) may comprise a V-fold configured to extend a path length of the one or more laser beams. In some embodiments, at least one of the one or more sensing media (120) may comprise a V-fold configured to extend a path length of the one or more laser beams.

In some embodiments, the open laser cavity (130) may further comprise one or more optical elements. In some embodiments, the one or more optical elements may comprise nonlinear crystals, frequency-selective elements, saturable absorbers, electro-optic devices, or a combination thereof. In some embodiments, the imaging system of the present invention may be capable of analyzing objects 0 nm or more away from the system.

In some embodiments, the one or more laser beams may comprise a plurality of laser portions. In some embodiments, the one or more laser beams may comprise a plurality of laser beams. The one or more gain chips (110) may comprise a plurality of gain chips such that each gain chip is configured to generate a laser beam of the plurality of laser beams. In some embodiments, the one or more laser beams may comprise a laser beam having a higher-order laser mode profile. In some embodiments, the one or more sensing media (120) may comprise a plurality of sensing media such that one or more laser portions of the plurality of laser portions are configured to interact with each sensing medium of the plurality of sensing media. In some embodiments, the one or more sensing media (120) may comprise a sensing medium comprising a plurality of laser interaction locations such that one or more laser portions of the plurality of laser portions are configured to interact with each laser interaction location of the plurality of laser interaction locations.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A laser device (100) for measuring one or more properties of one or more objects (200), the device (100) comprising:
a. an open laser cavity (130) configured to contain one or more laser beams, each laser beam comprising one or more laser portions, the open laser cavity (130) comprising:
i. one or more gain chips (110) disposed within the open laser cavity (130), configured to generate the one or more laser beams; and
ii. one or more sensing media (120) disposed within the open laser cavity (130) and optically in line with the one or more gain chips (110), each sensing medium comprising a proximal end (122) having a transmissive coating and a distal end (124) having a reflective coating, configured to accept the one or more laser portions of the one or more laser beams, measure the one or more properties of the one or more objects (200) based on illumination by the one or more laser portions, and reflect the one or more laser portions of the one or more laser beams back to the one or more gain chips (110).

2. The device (100) of claim 1, wherein at least one of the one or more gain chips (110) comprises a vertical external-cavity surface-emitting laser.

3. The device (100) of claim 1, wherein the one or more sensing media (120) are configured to interact with the one or more laser portions of the one or more laser beams and interact with an external force such that a measurable change in the one or more laser portions takes place.

4. The device (100) of claim 3, wherein at least one of the one or more sensing media (120) comprise a nitrogen-vacancy diamond, nitrogen-vacancy silicon carbide, or a combination thereof.

5. The device (100) of claim 1, wherein at least one of the one or more gain chips (110) comprises a V-fold configured to extend a path length of the one or more laser portions.

6. The device (100) of claim 1, wherein at least one of the one or more sensing media (120) comprises a V-fold configured to extend a path length of the one or more laser portions.

7. The device (100) of claim 1, wherein the open laser cavity (130) further comprises one or more optical elements.

8. The device (100) of claim 1, wherein the one or more laser beams comprise a plurality of laser beams, wherein the one or more gain chips (110) comprise a plurality of gain chips such that each gain chip is configured to generate a laser beam of the plurality of laser beams.

9. The device (100) of claim 1, wherein the one or more laser beams comprise a laser beam having a higher order laser mode profile.

10. The device (100) of claim 1, wherein the one or more sensing media (120) comprise a plurality of sensing media such that at least one of the one or more laser portions are configured to interact with each sensing medium of the plurality of sensing media.

11. The device (100) of claim 1, wherein at least one of the one or more sensing media (120) comprises a sensing medium comprising a plurality of laser interaction locations such that at least one of the one or more laser portions is configured to interact with each laser interaction location of the plurality of laser interaction locations.

12. A laser device (100) for measuring one or more properties of one or more objects (200) adjacent to the device (100), the device (100) comprising:
a. an open laser cavity (130) having a first end and a second end, the open laser cavity (130) comprising:
i. a plurality of gain chips (110) disposed at the first end of the open laser cavity (130), configured to generate one or more laser beams comprising a plurality of laser portions; and
ii. a plurality of sensing media (120) disposed at the second end of the open laser cavity (130) and optically in line with the plurality of gain chips (110), each sensing medium comprising a proximal end (122) having a transmissive coating and a distal end (124) having a reflective coating, configured to accept the one or more laser beams, measure the one or more properties of the one or more objects (200) based on illumination by the one or more laser beams, and reflect the one or more laser beams back to the plurality of gain chips (110);
wherein one or more laser portions of the plurality of laser portions are configured to interact with each sensing medium of the plurality of sensing media (120).

13. The device (100) of claim 12, wherein at least one of the plurality of gain chips (110) comprises a vertical external-cavity surface-emitting laser.

14. The device (100) of claim 12, wherein the plurality of sensing media (120) are configured to interact with at least one portion of the plurality of laser portions of the one or more laser beams and interact with an external force such that a measurable change in the at least one portion takes place.

15. The device (100) of claim 12, wherein at least one of the plurality of gain chips (110) comprises a V-fold configured to extend a path length of the one or more laser beams.

16. The device (100) of claim 12, wherein at least one of the plurality of sensing media (120) comprises a V-fold configured to extend a path length of the one or more laser beams.

17. The device (100) of claim 12, wherein the open laser cavity (130) further comprises one or more optical elements.

18. A laser device (100) for measuring one or more properties of one or more objects (200) adjacent to the device (100), the device (100) comprising:
a. an open laser cavity (130) having a first end and a second end, the open laser cavity (130) comprising:
i. a gain chip (110) disposed at the first end of the open laser cavity (130), configured to generate a laser beam having a higher order laser mode profile, the laser beam comprising a plurality of laser portions; and
ii. one or more sensing media (120) disposed at the second end of the open laser cavity (130) and optically in line with the gain chip (110), each sensing medium comprising a proximal end (122) having a transmissive coating and a distal end (124) having a reflective coating, configured to accept the one or more laser beams, measure the one or more properties of the one or more objects (200) based on illumination by the one or more laser beams, and reflect the one or more laser beams back to the gain chip (110);
wherein one or more laser portions of the plurality of laser portions are configured to interact with each sensing medium of the one or more sensing media (120).

19. The device (100) of claim 18, wherein at least one of the plurality of gain chips (110) comprises a vertical external-cavity surface-emitting laser.

20. The device (100) of claim 18, wherein the one or more sensing media (120) are configured to interact with at least one portion of the plurality of laser portions and interact with an external force such that a measurable change in the at least one portion takes place.
